(19) United States
(12) Patent Application Publication
(10) Pub. No.: US 2005/0186137 A1
(43) Pub. Date: Aug. 25, 2005

(54) Novelty-induced hypophagia-based assays for identifying agents to treat anxiety and depression

(76) Inventors: Rene Hen, Tenafly, NJ (US); Stephanie Dulawa, New York, NY (US)

Correspondence Address: Cooper and Dunham LLP, 1185 Avenue of the Americas, New York, NY, 10036, US

(21) Appl. No.: 10/977,911

(22) Filed: Oct. 29, 2004

Related U.S. Application Data
(60) Provisional application No. 60/516,666, filed on Oct. 31, 2003.

(51) Int. Cl. A61K 49/00

(52) U.S. Cl. 424/9.1

(57) ABSTRACT

This invention provides a method for determining whether an agent reduces anxiety and/or depression in a depressed, non-human subject. This invention also provides methods for treating anxiety and depression in subjects afflicted with those disorders comprising administering a therapeutically effective amount of an anxiety-reducing or depression-reducing agent identified by the instant method.

This application claims the benefit of U.S. Provisional Application No. 60/516,666, filed Oct. 31, 2003, the contents of which are hereby incorporated by reference.

Throughout this application, various publications are referenced. Full citations for these publications may be found immediately preceding the claims. The disclosures of these publications are hereby incorporated by reference into this application in order to more fully describe the state of the art as of the date of the invention described and claimed herein.

The invention was made with support under NIH Grant Nos. R01 DA09862, R01 MH068542-1, and P50 MH50733 from the National Institutes of Health. Accordingly, the U.S. Government has certain rights in this invention.

### BACKGROUND OF THE INVENTION

Animal models sensitive to chronic, but not acute, antidepressant activity are much sought after tools which have remained elusive. While acute antidepressant treatment results in increases in synaptic monoamines within minutes to hours, weeks of sustained treatment are required for induction of therapeutic effects (Blier, 2003). Although some acute paradigms, such as the forced swim and tail suspension tests, provide good assay models for screening novel antidepressant compounds, few models have been developed in which chronic, but not acute, antidepressant treatment alters behavior (Cryan et al., 2002a). Of those, none have strong predictive validity and high reliability while maintaining ease of use (Cryan et al., 2002a).

### SUMMARY OF THE INVENTION

This invention provides a method for determining whether an agent reduces anxiety and/or depression in a depressed, non-human subject, comprising (a) determining the subject's rate of engagement in a pleasurable activity in a novel environment, wherein at the time the subject is introduced to the novel environment, the subject has had the agent administered to it over a suitable period of time, (b) comparing the subject's rate of engagement determined in step (a) with the subject's rate of engagement in the pleasurable activity determined prior to the subject's introduction to the novel environment when the subject is in a familiar environment and has had the agent administered to it for a suitable period of time, so as to determine the difference in rates of engagement, and (c) comparing the difference in rates of engagement determined in step (b) with the difference in rates of engagement determined in a depressed, non-human subject to which the agent has not been administered, whereby the difference determined in step (b) being less than the difference determined in the absence of the agent indicates that the agent reduces anxiety and/or depression in a depressed, non-human subject.

This invention also provides a method of treating anxiety in a subject afflicted with that disorder comprising administering to the subject a therapeutically effective amount of an anxiety-reducing agent identified by the instant method.

Finally, this invention provides a method of treating depression in a subject afflicted with that disorder comprising administering to the subject a therapeutically effective amount of a depression-reducing agent identified by the instant method.

### BRIEF DESCRIPTION OF THE FIGURES

FIG. 1

Motor activity. Total locomotor activity assessed in the OF test on three consecutive days is shown for Balb/cJ, 129SvEv, C57BL/6J, and DBA/2J mice receiving 0, 5, or 10 mg/kg/day chronic fluoxetine. Values are means ±SEM. *P<0.05 vs. control group with ANOVA.

FIG. 2

Modified Forced Swim Test. Total time engaged in (a) swimming or (b) immobility are shown for Balb/cJ, 129SvEv, C57BL/6J, and DBA/2J mice receiving 0, 5, or 10 mg/kg/day chronic fluoxetine. Values are means ±SEM. *P<0.05 vs. control group with ANOVA.

FIG. 3

Plasma Fluoxetine Levels. Plasma fluoxetine levels are shown in ng/ml for Balb/cJ mice receiving 10, 18, or 25 mg/kg/day chronic fluoxetine. The area in grey bars shows plasma fluoxetine levels for patients taking 20-80 mg Prozac per day.

FIG. 4

Open Field Test. Log transformed data for OF measures are shown for Balb/cJ mice receiving 0, 10, 18, and 25 mg/kg/day chronic fluoxetine. Log of total locomotor activity (a), log of the distance traveled in the center (b), log of entries into the center (c), and the log of center/total distance traveled (d) are shown in cm. Untransformed data are shown in insets. Values are means ±SEM. *P<0.05 vs. control group with ANOVA.

FIG. 5

Novelty-Induced Hypophagia. The effects of a novel cage on latency to consume, and the amount consumed, of a familiar and palatable snack are shown for Balb/cJ mice. The difference in latency to consume in the home vs. a novel cage (a), the latency to consume in the home and a novel cage (b), and the amount consumed in the first 5 min in the home and a novel cage (c) are shown for Balb/cJ mice receiving 0, 10, 18, or 25 mg/kg/day chronic fluoxetine. Values are means ±SEM. *P<0.05 vs. control group with ANOVA.

FIGS. 6A-6E

Modified Forced Swim Test. Total time engaged in swimming (a) or immobility (b) are shown for Balb/cJ mice receiving 0, 10, 18, or 25 mg/kg/day chronic fluoxetine. Values are means ±SEM. *P<0.05 vs. control group with ANOVA. Forced swim test. Total time engaged in swimming (c), immobility (d), or climbing (e) are shown for BALB/c mice receiving 0, 10, 18, or 25 mg/kg/day (n=15 per group). Values are means ±SEM. *P<0.05 vs. control group with ANOVA.

FIG. 7

Prepulse Inhibition. Percent PPI (a) or startle magnitude (collapsed for blocks two and three) (b) values are shown for Balb/cJ mice receiving either 0, 10, 18, or 25 mg/kg/day chronic fluoxetine and an acute treatment of either 0 or 10 mg/kg RU24969. PPI was evaluated using 3, 6, and 12 dB prepulse intensities. Values are means ±SEM. *P<0.05 vs. control group for acute treatment with ANOVA.

### DETAILED DESCRIPTION OF THE INVENTION

### DEFINITIONS

As used in this application, except as otherwise expressly provided herein, each of the following terms shall have the meaning set forth below.

As used herein, “administered” shall mean delivered in a manner which is effected or performed using any of the various methods and delivery systems known to those skilled in the art. An agent can be administered, for example, intravenously, orally, via implant, transmucosally, transdermally, intramuscularly, or subcutaneously. An agent can also be “administered”, for example, once, a plurality of times, and/or over one or more extended periods.

As used herein, “agent” shall include, without limitation, an organic or inorganic compound, a nucleic acid, a polypeptide, a lipid, a carbohydrate or a physical stimulus. Agents include, for example, agents which are known with respect to structure and/or function, and those which are not known with respect to structure or function.

As used herein, “anxiety” shall refer to a mental state, such as a mental disorder, in a subject that creates a state of uneasiness or apprehension resulting from the anticipation of a realistic or imagined threatening event or situation.

As used herein, “subject” shall mean any animal, for example, a mouse, rat, guinea pig, dog, cat, rabbit, non-human primate or human.

As used herein, the “depression” shall mean a mental state, such as a mental disorder, in a subject that results in feelings of sadness and/or despondency, occasionally accompanied by a reduction in the subject's activity or energy level.

As used herein, a “familiar” environment is an environment to which a subject has been previously exposed. A “novel” environment is an environment to which a subject has not been previously exposed.

As used herein, a “pleasurable activity” means any activity in which a subject would willingly engage in without either motivation or coercion from an external stimulus. One example of a pleasurable activity is the consumption of an enjoyable meal, in which the subject would willing engage without motivation (e.g. pleasurable electrical stimulus) or coercion (e.g. painful electrical stimulus) from an external stimulus.

As used herein, “rate of engagement” in an activity can be measured, for example, by measuring the total amount of time spent engaged in the activity, elapsed time period between the consecutive engagements in the activity, and the intensity of engagement in the activity.

As used herein, “reducing” anxiety or depression in a subject shall mean either lessening the severity of the disorder, or eliminating the disorder entirely. Furthermore, an agent is effective in “reducing” anxiety or depression when a subject displays for example at least a 10% increase in rates of engagement after the agent is administered. In the preferred embodiment, the subject displays at lest a 20%, 30%, 40% or 50% increase in rates of engagement after the agent is administered.

### EMBODIMENTS OF THE INVENTION

This invention provides a method for determining whether an agent reduces anxiety and/or depression in a depressed, non-human subject, comprising (a) determining the subject's rate of engagement in a pleasurable activity in a novel environment, wherein at the time the subject is introduced to the novel environment, the subject has had the agent administered to it over a suitable period of time, (b) comparing the subject's rate of engagement determined in step (a) with the subject's rate of engagement in the pleasurable activity determined prior to the subject's introduction to the novel environment when the subject is in a familiar environment and has had the agent administered to it for a suitable period of time, so as to determine the difference in rates of engagement, and (c) comparing the difference in rates of engagement determined in step (b) with the difference in rates of engagement determined in a depressed, non-human subject to which the agent has not been administered, whereby the difference determined in step (b) being less than the difference determined in the absence of the agent indicates that the agent reduces anxiety and/or depression in a depressed, non-human subject.

In one embodiment of the instant method, the subject is a mammal, such as a mouse. In a preferred embodiment, the mouse is a Balb/cJ mouse.

In another embodiment of this method, the agent to be tested is selected from the group consisting of a MAO inhibitor, a NK1 antagonist, a CRF1 antagonist, a vasopression1B antagonist, a glucocorticoid receptor antagonist, a 5-HT1B receptor antagonist and a lithium mood stabilizer. In another embodiment, the agent is selected from the group consisting of desipramine, venlafaxine, reboxetine and fluoxetine. In still another embodiment, the agent is an electroconvulsive shock or transcranial magnetic stimulation.

In another embodiment of the instant method, the subject's rate of engagement in the pleasurable activity is the speed with which the subject commences the activity. In yet another embodiment, the subject's rate of engagement in the pleasurable activity is the speed with which the subject carries out the activity once the activity has commenced.

In another embodiment of the instant method, the pleasurable activity is selected from the group consisting of eating, drinking, sexual activity, object exploration, and the receipt of a pleasurable electrical stimulus.

In another embodiment of the instant method, the environment is a cage. In a further embodiment, the novel and familiar environments differ from one another via a difference in bedding, lighting and/or noise level.

In another embodiment of this method, the difference in rates of engagement determined in step (c) is at least 50%. In a further embodiment, the difference in rates of engagement determined in step (c) is at least 100%. In a still further embodiment, the difference in rates of engagement determined in step (c) is at least 150%.

In another embodiment of this method, the suitable period of time is at least one week. In yet another embodiment, the suitable period of time is at least two weeks. In still another embodiment, the suitable period of time is at least four weeks.

In another embodiment of this method, the method further comprises the step of performing the method a plurality of times using different dosages of the agent and comparing the differences in rates of engagement, so as to determine which of the dosages used best reduces anxiety and/or depression in the subject.

In another embodiment of this method, the method further comprises the step of comparing the differences in rates determined in step (c) for a subject to which the agent has been administered with the differences in rates determined in step (c) for a subject to which fluoxetine has been administered in the same dose as the agent, so as to determine whether the agent reduces anxiety and/or depression as well as, or better than, fluoxetine.

In another embodiment of this method, the method further comprises the step of comparing (i) the minimum period of time over which the agent must be administered to reduce anxiety and/or depression in the subject with (ii) the minimum period of time over which fluoxetine must be administered to reduce anxiety and/or depression in the subject, so as to determine whether the agent reduces anxiety and/or depression in a subject via administration over a time period as short as, or shorter than, that required for fluoxetine to achieve the same result.

This invention further provides a method of treating anxiety in a subject afflicted with that disorder comprising administering to the subject a therapeutically effective amount of an anxiety-reducing agent identified by the instant method.

Finally, this invention further provides a method of treating depression in a subject afflicted with that disorder comprising administering to the subject a therapeutically effective amount of a depression-reducing agent identified by the instant method.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

### METHODS

Chronic models of antidepressant activity are a requisite tool for identifying the neural mechanisms underlying the antidepressant response, and for gaining insights into the pathophysiology of the disorders they treat. Depression and anxiety disorders are burdensome conditions with lifetime prevalence rates of approximately 7-20% (R M Hirschfeld, 2002) and 11-27% (KR Merikanagas, 2002), respectively. The selective serotonin-reuptake inhibitors (SSRIs) are unique in that they can effectively treat virtually all categories of anxiety and depressive disorders. Due to the broad therapeutic potential of SSRIs, the prototypical SSRI, fluoxetine (Prozac), was used to develop novel animal models. A hypothesis was developed that highly anxious or emotional mouse strains, such as BALB/cJ and DBA/2J (Robertson, 1979; Bouwknecht and Paylor 2003; Tang et al., 2002), might exhibit behavioral alterations to chronic fluoxetine treatment. Thus, the effects of chronic fluoxetine treatment in four inbred mouse strains were assessed.

For medical disorders of unknown pathophysiology or genetic etiology, such as anxiety and affective disorders, the emerging approach to developing an animal model is to model a single symptom or aspect of the disorder. Geyer and Markou [Geyer, 1995 #73; Geyer, 2002 #117] suggest that the only criteria required for the initial use of a new model is that the behavioral measure is robust and reliable within and between laboratories and has strong predictive validity. Because anxiety, depression, and sensorimotor gating are all domains affected by chronic fluoxetine treatment in humans, the paradigms used provided operational measures of each of these variables. Since the Balb/cJ strain showed sensitivity to chronic fluoxetine treatment, the effects of both chronic and sub-acute fluoxetine treatment on this strain were assessed in the open field test, modified forced swim test, novelty-induced hypohagia test, and the prepulse inhibition paradigm (PPI).

The open field test is a classical and well-validated paradigm in which anxiety can be assessed as avoidance of the center (Welker, 1957; Belzung and Le Pape, 1994; Prut and Belzung, 2003). A new paradigm termed novelty-induced hypohagia (NIH) was developed, based on the finding that rodents exhibit reduced consumption of a familiar food when exposed to the stressor of a novel environment (Shepard and Broadhust, 1982; Bodnoff et al., 1988). The forced swim test is an assay model with high predictive validity for antidepressant compounds. Sensorimotor gating is a form of central nervous system inhibition that suppresses unwanted neural activity in the sensory, cognitive, or motor domains (Geyer and Braff, 1987; Perry et al., 1999; Geyer et al., 2001). PPI refers to the reduction in startle amplitude that results when an abrupt startling stimulus is preceded 30-500 msec by a non-startling prepulse (Graham, 1975) and is an operational measure of sensorimotor gating.

Subjects

Adult male mice of four strains: BALB/cJ, DBA/2J, C57BL/6J, (Jackson Laboratories, Bar Harbor, Me.), and 129S6/SvEvTac (Taconic Farms, Germantown, N.Y.) were subjects. BALB/cJ, and DBA/2J strains were selected for their anxious phenotypes; the C57BL/6J and 129SvEv strains were selected for their relatively non-anxious phenotypes. The C57BL/6J strain was also selected based on its frequency of use; the 129SvEv strain was also selected because embryonic stem cells for knockout mice are often derived from this strain. Mice were maintained on a 12 L:12 D schedule (lights on at 0600 h) and were housed in groups of five with same-type mice. Food and water were provided ad libitum. Behavioral testing occurred during the light phase between 0600 and 1800 h. All animal testing was conducted in accord with the NIH laboratory animal care guidelines, and with IACUC approval.

Drugs

Fluoxetine was a generous gift from Lilly (Indianapolis, Ind.), and was delivered ad libitum in the drinking water. Fluoxetine was dissolved in tap water and changed weekly. For initial strain comparison studies, daily drinking was measured and the concentration of fluoxetine solutions were adjusted to deliver 0, 5, or 10 mg/kg/day of fluoxetine to each strain. For subchronic and chronic studies in BALB/cJ mice, 80-mg/L, 160 mg/L, and 240 mg/L fluoxetine was given, and drinking was recorded throughout the study. Fluoxetine plasma levels were determined by liquid chromatography with fluorescence detection (Suckow et al., 1992) for chronically treated Balb/cJ mice. 5-methoxy-3(1,2,3,6)tetrahydropyridin-4-yl-1H-indole (RU24969) was obtained from Tocris Cookson (Ellisville, Mo.), dissolved in saline, and delivered i.p. (5 ml/kg) with 26 gauge needles. Both drugs were protected from light.

Experiments

For the strain comparison study, 36 male mice of four strains (BALB/cJ, DBA/2J, C57BL/6J, 129SvEv) were subjects for a total of 144 mice. Mice were tested in the OF beginning on day 30 or 33 of chronic fluoxetine treatment. Mice were tested for 15 min on three consecutive days to maximize activity levels. Mice were then tested in the modified FST on day 36 or 37.

For chronic fluoxetine studies in BALB/cJ mice, 60 male BALB/cJ mice were tested in the OF for 30 min on days 19 or 20 of chronic fluoxetine treatment. Mice were then pretested and tested in the modified FST on days 21 and 22, respectively. Animals were then allowed to adapt to being singly housed, and were trained for NIH on days 25-27. Home cage and novel cage testing occurred on days 28 and 29, respectively. Mice were then assessed for PPI on days 34 or 35.

Studies of subchronic fluoxetine were conducted using separate groups of 60 male BALB/cJ mice to prevent fluoxetine treatment from exceeding one week. One group was tested in the OF after one day of subchronic fluoxetine treatment. Mice were then pretested after 6 and tested after 7 days of subchronic fluoxetine treatment in the modified FST. A second group of BALB/cJ mice began fluoxetine treatment after being singly housed. They were then tested in the NIH test after 4 (home) and 5 (novel) days of subchronic fluoxetine treatment. A third group was tested for PPI after one or two days of subchronic fluoxetine treatment.

Apparatus and Procedures

Open field test: Motor activity was quantified in four Plexiglas OF boxes 43 x 43 cm (MED Associates, Georgia, Vt.). Two sets of 16 pulse-modulated infrared photobeams were placed on opposite walls 2.5-cm apart to record x-y ambulatory movements. Activity chambers were computer interfaced for data sampling at 100-ms resolution. The computer defined grid lines that divided each OF into center and surround regions, with each of four lines being 11 cm from each wall. Dependent measures were the number of entries into the center, the distance traveled in the center, total time spent in the center, and distance traveled in the center divided by total distance traveled. Overall motor activity was quantified as the total distance traveled (cm).

Novelty induced hypophagia test: Mice were singly housed several days before training began. For three consecutive days of training, mice were presented with diluted (1:3; milk:water) sweetened condensed milk (Carnation) in pipettes in the home cage for 30 min. Testing occurred on days 4 and 5 when mice were presented with the milk, and latency to drink and the volume consumed were recorded every five minutes for 30 min. On day 4, testing occurred in the home cage under dim lighting (approx. 50 lux). Mice were briefly removed from the cage to position pipettes, and testing began when mice were returned to their cages. On day 5, mice were placed into new cages of the same dimensions without shavings under bright lighting (approx. 1200 lux). White paper was placed under cages to enhance aversiveness on day 5.

Modified forced swim test: Mice were placed into 19 cm diameter plastic buckets which were 23 cm deep filled with 23°-25° C. water, and videotaped for six minutes. Only the last four minutes were scored for four measures: swimming, immobility, climbing, or other. The predominant behavior was recorded every 5 sec. For chronic and subchronic fluoxetine studies in BALB/cJ mice, mice were pre-exposed to the FST for 6 min 24 hours before test day to increase sensitivity for detecting antidepressant behavioral effects (Borsini et al., 1989).

Prepulse Inhibition: Startle experiments were performed in MED-Associates chambers. Nonrestrictive Plexiglas cylinders 5 cm in diameter rested on a Plexiglas platform with a transducer amplifier. The sound-dampening isolation chambers were ventilated, well-lit, and equipped with an acoustic stimulator. Data acquisition was performed using Med-Associates software.

Five different trial types were presented in the test session: a 40-msec broadband 120 dB burst (PULSE ALONE trial); three different PREPULSE+PULSE trials in which 20-msec long 3 dB (pp3), 6 dB (pp6), or 12 dB (pp12) stimuli above a 70 dB background preceded the 120 dB pulse by 100 msec (onset to onset), and a NO STIMULUS trial, in which only the background noise was presented. Trials were presented in a pseudo-random order. A variable interval of 10-20 seconds separated trials.

The test session consisted of 62 test trials. The session began with a 10 min acclimation period, followed by four consecutive blocks of test trials. Blocks one and four consisted of six consecutive PULSE ALONE trials, while blocks two and three contained six PULSE ALONE trials, five pp3+p120 trials, five pp6+p120 trials, five pp12+p120 trials, and four NO STIMULUS trials. Analyses were performed using average values for each trial type.

Statistical Analysis

Open field test: For Experiment 1, two-factor ANOVAs with fluoxetine as a between-subject factor and day as a within-subject factor were applied to five measures for each strain: time spent in the center, distance traveled in the center, center distance/total distance traveled, entries into the center, and total distance traveled. Main effects of fluoxetine or significant interactions of fluoxetine and day were resolved using post-hoc ANOVAs with adjusted p-values and/or Newman-Keuls post-hoc tests. Subchronic and chronic experiments in Balb/cJ mice were analyzed using one-way ANOVAs with fluoxetine as a between-subjects factor and Newman-Keuls post-hoc tests for the same variables. Because data deviated substantially from a normal distribution due to low activity levels of Balb/cJ mice, analyses were applied to log transformed data.

When significant differences in locomotor activity were observed in the OF, ANCOVAs were applied to center measures with locomotor activity as a covariate. Remaining significant effects of fluoxetine indicate independence between measures of locomotor activity and center measures.

Novelty induced hypophagia test: For latency scores, a maximum cut-off of 600 sec was used. Two-factor ANOVAs with home/novel cage condition as a within-subjects factor and fluoxetine as a between-subjects factor were applied to latency values. Main effects of fluoxetine or significant interactions of fluoxetine and home/novel cage condition were resolved using post-hoc ANOVAs with adjusted p-values and/or Newman-Keuls post-hoc tests. For consumption data, a three-factor ANOVA with block (5 min intervals) and home/novel cage condition as within-subjects factors and fluoxetine as a between-subjects factor was used to assess whether the novel cage was sufficiently anxiogenic to reduce consumption. Two-factor ANOVAs with block as a within-subjects factor and fluoxetine as a between-subjects factor were applied separately to consumption values for the home and the novel cage. Main effects of fluoxetine or significant interactions were resolved using post-hoc ANOVAs with adjusted p-values and/or Newman-Keuls post-hoc tests.

Modified forced swim test: Two-factor ANOVAs with fluoxetine as a between-subjects factor and block (1 min intervals) as a within-subject factor were applied to three different measures: swimming, immobility, and climbing. Significant main effects of fluoxetine and interactions were resolved using post-hoc ANOVAs with adjusted p-values, and/or Newman-Keuls post-hoc tests.

Prepulse Inhibition: PPI was calculated as the averaged startle magnitude caused by PULSE ALONE trials from blocks two and three, minus the averaged startle magnitude caused by the prepulse-pulse trials, all divided by the averaged PULSE ALONE values from blocks two and three. The resulting value was multiplied by 100 and presented as a percent PPI value. Four-factor ANOVAs with fluoxetine and RU24969 as between subjects factors and prepulse intensity and block as within subjects variables assessed percent PPI levels. Interactions were resolved using post-hoc ANOVAs with adjusted p-values, and/or Newman-Keuls post-hoc tests.

### RESULTS

Strain Comparison Studies

Open Field Test

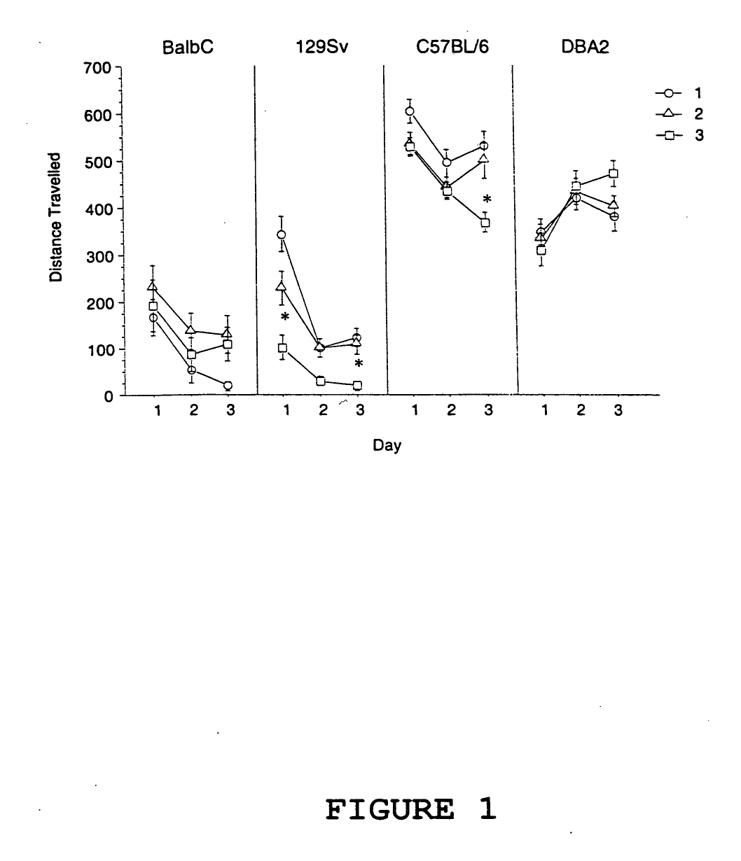

The OF test is a classical approach/avoidance paradigm in which the novel environment concurrently evokes both anxiety and exploration (Welker, 1957; Belzung and Le Pape, 1994; Dulawa et al., 1999; Prut and Belzung, 2003). Four strains of mice, BALB/cJ, DBA/2J, C57BL/6J, and 129SvEv, were tested on three consecutive days in the OF to assess locomotion and anxiety measures. ANOVAs revealed that BALB/cJ mice showed no changes in locomotor activity, while C57BL/6J and 129SvEv mice showed reductions in locomotor activity, and DBA/2J mice showed a trend towards increased locomotor activity in response to chronic fluoxetine treatment (FIG. 1). The 10 mg/kg/day dose of fluoxetine reduced locomotor activity in 129SvEv mice [F(4,58)=4.15, P=0.005] relative to both other doses on day 1, and relative to the 5 mg/kg/day dose on day 3. The 10 mg/kg/day dose of fluoxetine reduced locomotor activity overall in C57BL mice [F(2,33)=3.30, P=0.05] relative to controls. DBA/2J mice showed a trend [F(4,62)=2.40, P=0.06] for the 10 mg/kg/day dose of fluoxetine to increase locomotion relative to controls on day 3.

None of the four strains exhibited increases in center entries, time in the center, distance traveled in the center, or center/total distance traveled in response to chronic fluoxetine treatment. In fact, chronic fluoxetine reduced center measures in C57BL/6J and 129SvEv mice, and produced no changes in BALB/cJ and DBA/2J mice. Both doses of fluoxetine (5 and 10 mg/kg/day) reduced time in the center relative to control in 129SvEv mice [F(2, 29)=4.90, P=0.01]. Additionally, both doses of fluoxetine reduced the distance traveled in the center [F(4,58)=3.11, P=0.02] and center entries [F(4,58)=4.08, P=0.006] relative to controls on day 1 in 129SvEv mice. C57BL/6J mice also showed an overall reduction in the number of entries into the center [F(2,33)=8.00, P=0.002] for both doses of fluoxetine relative to control. The decrease in center measures exhibited by C57BL/6J and 129SvEv mice in response to drug was likely related to decreased locomotor activity. Indeed, ANCOVAs revealed that drug no longer had a significant effect on center measures when data were analyzed using locomotor activity as a covariate. Furthermore, no effects of chronic fluoxetine on center/total distance traveled were found in any strain.

Modified Forced Swim Test

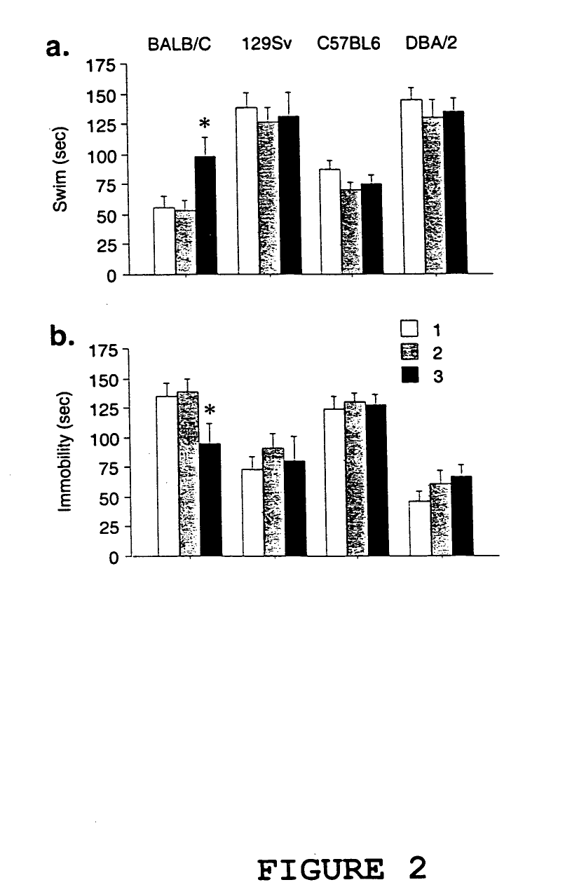

The FST is an assay model with high predictive validity for antidepressant compounds. The modified FST results from several simple procedural modifications including increased water depth and scoring behavior with a time sampling technique (see methods) that render the traditional FST sensitive to SSRIs (Cryan et al., 2002a). The BALB/cJ was the only strain showing sensitivity to chronic treatment with fluoxetine (FIG. 2). BALB/cJ mice treated with the 10 mg/kg/day dose of fluoxetine showed increased swimming [F(2, 33)=4.37, P=0.02] and decreased immobility [F(2, 33)=3.52, P=0.04] relative to the 5 mg/kg/day dose and controls. No effects on climbing were observed.

Chronic vs. Subchronic Effects of Fluoxetine in BALB/cJ Mice

Serum Fluoxetine Levels

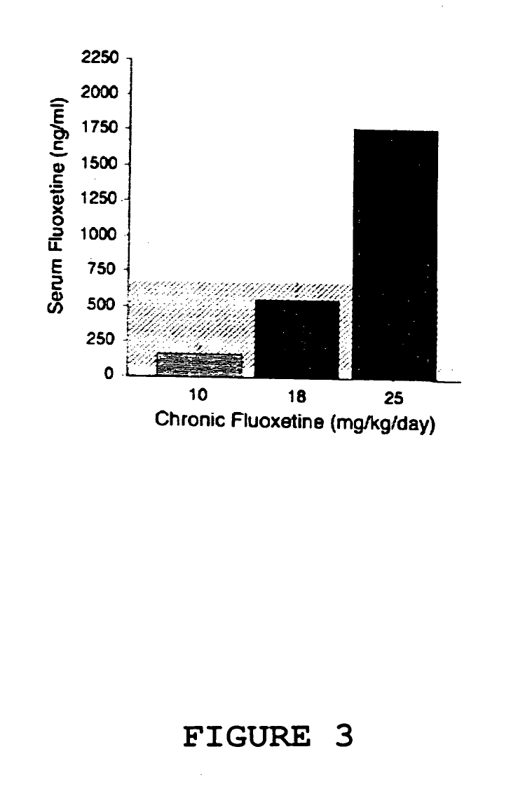

Serum fluoxetine levels were assessed in Balb/cJ mice receiving chronic fluoxetine treatment (0, 80, 160, 240 mg/L drinking water) (FIG. 3). The present findings are consistent with reports that fluoxetine exhibits nonlinear kinetics, indicated by a disproportionate increase in its blood concentrations after dose escalation (Caccia et al., 1990; Hiemke and Hartter, 2000). Based on the concentration of fluoxetine administered and the amount animals drank throughout the experiment, actual fluoxetine doses were 0, 10, 18, and 25 mg/kg/day. Serum fluoxetine levels for the 10 mg/kg/day dose (170.3±57.8 ng/ml) were toward the bottom of the range of plasma levels found in patients taking 20-80 mg/day Prozac (100-700 ng/ml) (Koran et al., 1996) (Suckow, personal communication). Serum levels for the 18 mg/kg/day dose (563.4±118.4 ng/ml) were toward the high end of this range. The 25 mg/kg/day dose of fluoxetine resulted in serum levels (1779.2±364.9 ng/ml) that were over twice the maximum of the human range. Serum levels for norfluoxetine (ng/ml) were 205.3±53.8, 495.4±99.0, and 1132±98.1 for the 10, 18, and 25 mg/kg/day doses, respectively. The ratio of plasma fluoxetine to norfluoxetine observed here is similar to that reported for humans (Koran et al., 1996).

Open Field Test

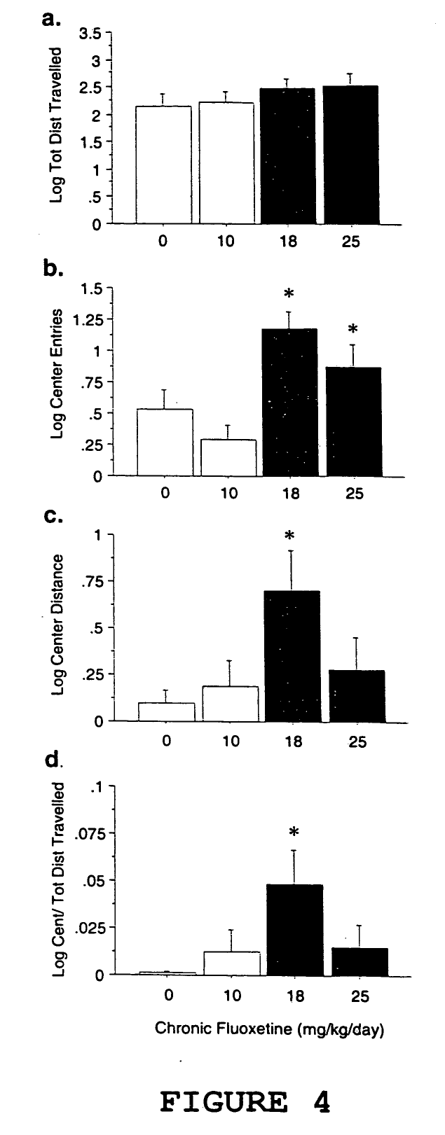

BALB/cJ mice treated chronically with fluoxetine were tested in the OF for 30 min. As in the strain comparison study, fluoxetine did not alter total locomotor activity in BALB/cJ mice [F(3,56)=1.36, P=0.27]. However, for the log of center entries [F(3,56)=6.85, P=0.0005], the log of center distance [F(3,56)=2.89, P=0.04], and the log of center distance/log total distance traveled [F(3,56)=2.69, P=0.05], mice treated with 18 mg/kg/day fluoxetine exhibited significantly higher values than controls (FIG. 4). For the log of center entries, mice treated with 10 mg/kg/day fluoxetine also exhibited lower values than groups receiving 18 mg/kg/day and 25 mg/kg/day. Although the same trend was observed, there was no significant effect of drug on log of time in the center. Even without logarithmic transformation, mice treated with 18 mg/kg/day fluoxetine made more center entries [F(3,56)=3.07, P=0.04] and showed a trend for higher center/total distance values [F(3,56)=2.51, P=0.07] than controls, while total locomotor activity remain unaffected by all doses of drug [F(3,56)=0.86, P=0.47].

BALB/cJ mice treated subchronically (one day) with fluoxetine showed no differences in any measures in the OF, whether data were analyzed untransformed, or following logarithmic transformation (Table 1).

TABLE 1

| | | Subchronic | | | Chronic | | |
|---|---|---|---|---|---|---|---|
| | | df | F value | P-value | df | F value | P-value |
| Open Field | | | | | | | |
| Center Entries | Drug | 3.55 | 0.56 | 0.6470 | 3.56 | 6.85 | 0.0005* |
| Center Distance | Drug | 3.55 | 0.82 | 0.4890 | 3.56 | 2.89 | 0.0400* |
| Center/Tot | Drug | 3.55 | 0.65 | 0.5840 | 3.56 | 2.69 | 0.0500* |
| Distance | | | | | | | |
| Forced Swim Test | | | | | | | |
| Swimming | Drug | 3.56 | 1.26 | 0.297 | 3.56 | 3.20 | 0.0300* |
| Novelty Induced | | | | | | | |
| Hypophagia | | | | | | | |
| Latency | Drug × Cage | 3.52 | 0.57 | 0.6300 | 3.48 | 3.93 | 0.0100* |
| Home Consumption: | Drug | 15.260 | 0.88 | 0.5877 | 15.245 | 2.29 | 0.0050* |
| 0-5 min | | | | | | | |
| Novel Consumption: | Drug | 15.260 | 1.34 | 0.1770 | 15.240 | 2.10 | 0.0100* |
| 0-5 min | | | | | | | |
| Prepulse | | | | | | | |
| Inhibition | | | | | | | |
| PPI | Intensity × | 6.102 | 0.18 | 0.9831 | 6.102 | 3.00 | 0.0100* |
| | Drug × | | | | | | |
| | RU24969 | | | | | | |

Table 1 shows the effects of subchronic vs. chronic fluoxetine on dependent measures in Balb/cJ mice. All dependent measures showing a statistically significant effect of chronic fluoxetine were unaltered by subchronic fluoxetine.

Novelty-Induced Hypophagia

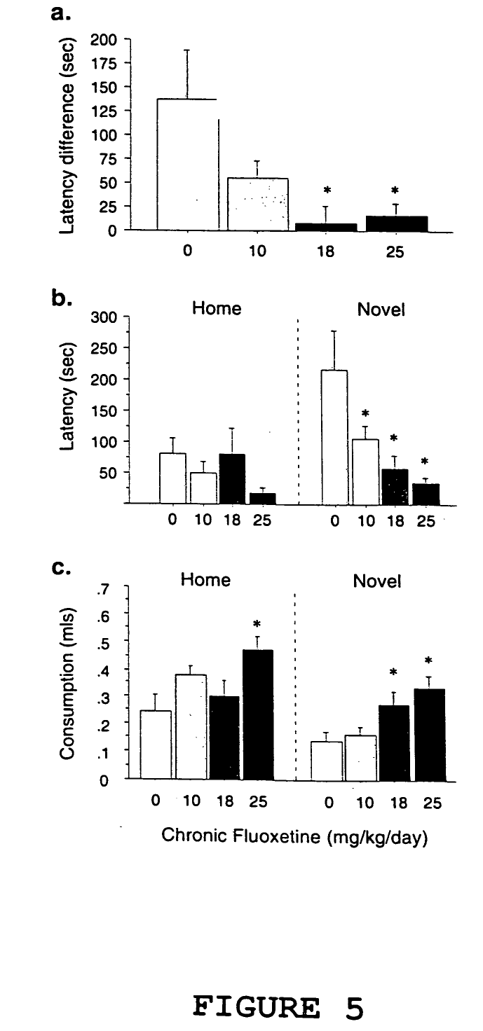

The NIH paradigm is based on the finding that rodents exhibit reductions in consumption of a familiar food when exposed to the stress of a novel environment (Shephard and Broadhurst, 1982; Bodnoff et al., 1988). Mice were assessed for the latency to consume and the amount consumed of a familiar and palatable snack in the home cage and a novel cage. Chronic fluoxetine treatment reduced the latency to drink milk in the novel, but not the home cage [F(3,48)=3.87, P=0.01 ] (FIG. 5a). In the novel cage, all three doses of fluoxetine reduced the latency to drink relative to control in a dose-related fashion. For latency difference scores (novel minus home), control animals exhibited significantly larger difference scores than animals receiving 18 mg/kg/day and 25 mg/kg/day fluoxetine (FIG. 5a).

Chronic fluoxetine treatment also attenuated another anxiety-related measure as assessed by consumption in the novel environment. Milk consumption was reduced overall in the novel environment compared to the home environment during the first five minutes [F(1,103)=11.86, P=0.0008], indicating that the novel environment was anxiety-provoking. Additionally, both the 18 mg/kg/day and 25 mg/kg/day doses of fluoxetine increased milk consumption compared to the 0 and 10 mg/kg/day doses in the first block [F(15,240)=2.10, P=0.01] in the novel cage (FIG. 5b). In the home cage, the 25 mg/kg/day dose of fluoxetine increased milk consumption compared to control in the first block [F(15,245)=2.29, P=0.005].

Subchronic (4-5 days) treatment with fluoxetine had no effect on any measure of latency. The overall latency to drink was increased in the novel cage [F(1,47)=4.33, P=0.04], suggesting that the novel cage was anxiogenic. Similarly, milk consumption was reduced in the novel environment compared to the home condition during the first five minutes [F(1,99)=9.62, P=0.003]. There was no effect of subchronic fluoxetine treatment on milk consumption. While ANOVA found a main effect of fluoxetine on consumption in the home cage [F(3,47)=3.57, P=0.02], post-hoc tests found only a trend for the 25 mg/kg/day dose to increase consumption relative to control (Table 1).

Modified Forced Swim Test

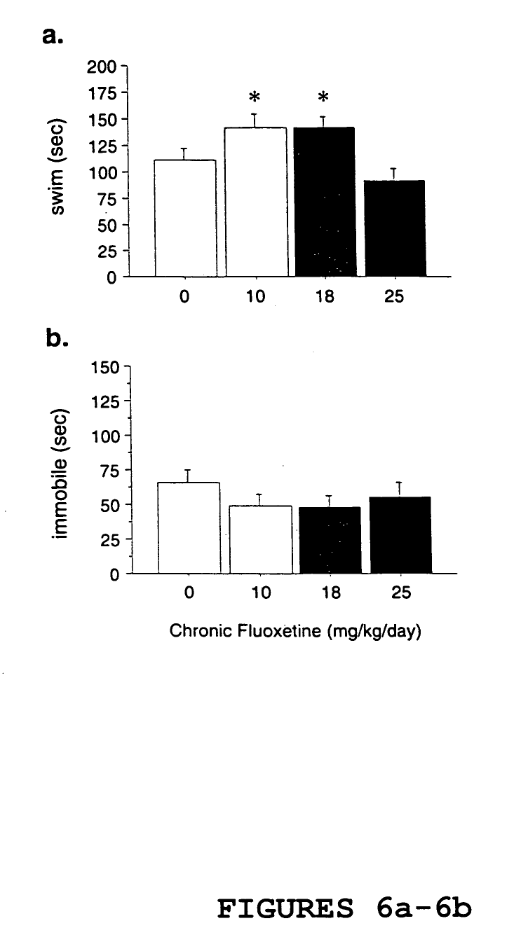

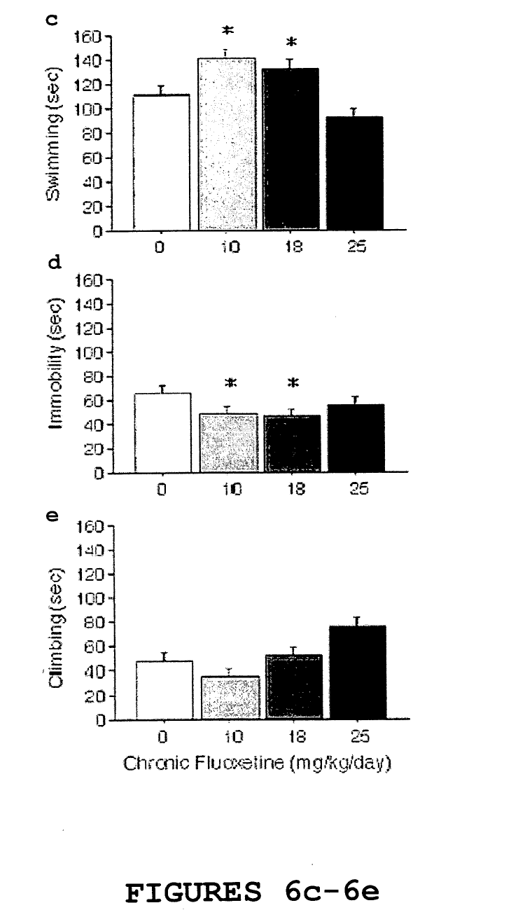

In the modified FST, SSRIs decrease immobility and increase swimming, while catecholaminergic agents decrease immobility and increase climbing (Cryan and Lucki, 2000; Cryan et al., 2002a; Cryan et al., 2002b). On test day, BALB/cJ mice treated chronically with 10 mg/kg/day and 18 mg/kg/day fluoxetine showed increases in swimming compared to mice treated with 0 and 25 mg/kg/day of fluoxetine [F(3,55)=4.25, P=0.009] (FIG. 6a). No significant effects on immobility were found, although a trend for fluoxetine to reduce immobility was observed (FIG. 6b). Mice treated with 25 mg/kg/day of chronic fluoxetine also showed increased climbing relative to all other doses [F(3,55)=3.48, P=0.02]. The increase in climbing induced by 25 mg/kg/day may have resulted from a loss of specificity for the serotonergic system of this high dose (FIG. 3).

Subchronic (7 days) treatment with fluoxetine had no effect on swimming or immobility (Table 1). The only effect was an increase in climbing induced by the 18 mg/kg/day dose relative to the 0 dose [F(3,52)=3.14, P=0.03]. Although catecholaminergic, but not serotonergic, agents are thought to increase climbing behavior, this hypothesis is based on results from acute, but not chronic drug studies (Cryan and Lucki, 2000; Cryan et al., 2002a; Cryan et al., 2002b).

Prepulse Inhibition

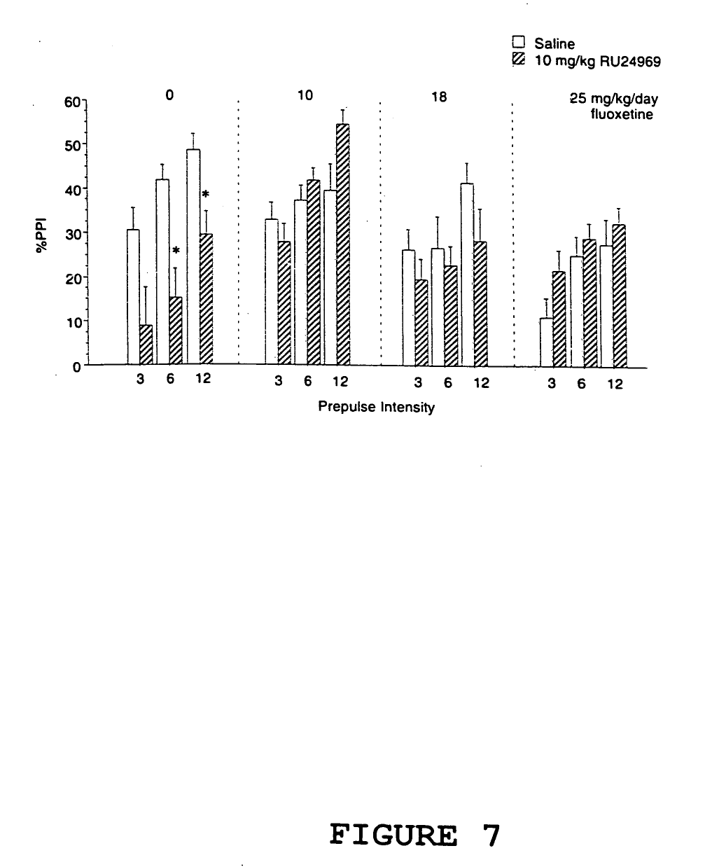

PPI is an operational measure of sensorimotor gating, and refers to the reduction in startle amplitude that results when an abrupt startling stimulus is preceded 30-500 msec by a non-startling prepulse (Graham, 1975; Geyer and Braff, 1987; Perry et al., 1999; Geyer et al., 2001). Control mice, but not mice treated with chronic fluoxetine, showed disruptions in PPI after receiving 10 mg/kg RU24969 [F(6,102)=3.00, P=0.01 ] (FIG. 7a). Post-hoc ANOVAs and Newman-Keuls post-hoc tests showed that RU24969 disrupted PPI in control mice, but not mice treated with any dose of fluoxetine at the 3 dB [F(3,110)=3.40, P=0.02], 6 dB [F(3,110)=2.55, P=0.06], and 12 dB [F(3,110)=3.88, P=0.01] prepulse intensities. In addition, RU24969-treated mice receiving 10 mg/kg/day fluoxetine showed increased PPI relative to RU24969-treated mice receiving 0 and 18 mg/kg/day fluoxetine at the 12 dB prepulse intensity [F(3,52)=4.93, P=0.004]. No effects of fluoxetine alone on PPI were found.

PPI were not confounded by effects of drugs on blocks two and three startle reactivity values, which are used to calculate PPI. Startle reactivity was increased overall by 10 mg/kg/day RU24969 [F(1,51)=9.98, P=0.003], but no interactions with chronic fluoxetine treatment were found (FIG. 7b).

Mice treated subchronically with fluoxetine (1-2 days) showed an overall effect for 10 mg/kg RU24969 to disrupt PPI [F(1,51)=13.86, P=0. 0005] and an interaction of RU24969 and prepulse intensity [F(2,102)=3.85, P=0.02]. Post-hoc ANOVAs showed that RU24969 disrupted PPI at each prepulse intensity. No interactions of RU24969 with fluoxetine were found (Table 1). Similarly, there were no effects of RU24969 or subchronic fluoxetine on startle reactivity in the second and third blocks.

### DISCUSSION

Four novel effects of chronic, but not subchronic, fluoxetine treatment on mouse behavior were tested. In the highly emotional Balb/cJ strain, chronic fluoxetine treatment increased center measures in the OF. In the NIH test, chronic fluoxetine treatment reduced the latency and increased the consumption of a palatable meal in a novel environment. In the modified FST, chronic fluoxetine treatment increased swimming behavior. Finally, chronic fluoxetine treatment also prevented PPI disruptions induced by the 5-HT1A/1B agonist RU24969. No effects of subchronic fluoxetine treatment (1-7 days) were observed (Table 1), suggesting that these models are specifically responsive to the chronic administration of fluoxetine.

The initial identification of the Balb/cJ strain as sensitive to chronic fluoxetine was critical for the development of the mouse models presented here. The hypothesis that highly anxious strains might exhibit sensitivity to chronic fluoxetine was based on reports that antidepressants can have profound mood-altering effects in depressed or anxious patients, but have few effects in healthy individuals (Barr et al., 1997; Gelfin et al., 1998; MA Geyer, 2002). Inducing deficits in animals that are cardinal features or symptoms of anxiety or depressive disorders may be essential for establishing models of antidepressant activity. For example, desmethylimipramine and low dose fluoxetine have no effect on intracranial self-stimulation reward thresholds unless animals have undergone drug withdrawal (Kokkinidis et al., 1980; Barr et al., 2002) or chronic stress (Markou et al., 1992; Moreau et al., 1992). Although preclinical models have used genetic, pharmacological, developmental, and environmental perturbations to induce various deficits, the use of a highly anxious strain, such as the Balb/cJ, offers unique advantages. Using an anxious strain avoids the time, effort, and potential variability of chronic stress paradigms, and offers a more unbiased approach for identifying novel therapeutic targets than models based on pharmacological perturbations. Furthermore, strain differences observed in the present models will permit the application of classic genetic tools to identify novel genes underlying fluoxetine responsiveness.

The strain comparison of the effects of chronic fluoxetine in the OF revealed that 129SvEv and C57BL/6J mice showed reduced locomotor activity to 10 mg/kg/day of fluoxetine. This sedative effect has been previously observed in the 129SvEv strain (data not shown). This reduction in locomotor activity could potentially confound other behavioral measures attempting to assess anti-anxiety or antidepressant effects of chronic fluoxetine. Thus, these two commonly used strains may be undesirable for studying the effects of chronic fluoxetine treatment. The Balb/cJ strain, however, exhibited no sedative effect and no trend towards increased locomotor activity, like the DBA/2J strain. The lack of effect of chronic fluoxetine on locomotion together with the increase in swimming observed in the strain comparison study indicated that the Balb/cJ strain was the most suitable strain for assessing the effects of chronic fluoxetine.

Anxiety measures of the OF test have been suggested to be insensitive to antidepressant treatment by a substantial literature (for review see Prut and Belzung, 2002). Here, Balb/cJ mice receiving 18 mg/kg/day chronic fluoxetine showed increases in center entries, distance traveled in the center, center/total distance traveled with no change in total locomotor activity. Subchronic treatment with fluoxetine was completely without effect (Table 1). Despite the low activity levels overall in these mice, these findings strongly suggest that 18 mg/kg/day of chronic fluoxetine decreases anxiety and/or increases exploration (Dulawa et al., 1999) in Balb/cJ mice. Although a large number of studies have found no effect of antidepressants in the OF (Prut and Belzung, 2002), few have evaluated the effects of chronic treatment in anxious animals. The effects of chronic antidepressant treatment on OF measures in Balb/cJ mice have not been previously reported.

The modified FST has strong predictive validity, high reliability, and is easy to use (Cryan et al., 2002a). However, acute antidepressant-like effects in the FST are likely to reflect the initial events elicited by antidepressants rather than the progressive changes that are likely to accompany chronic antidepressant treatment. Only one report has suggested that in rats, antidepressant-like effects of chronic, but not acute, antidepressants could be observed in the FST with low doses of antidepressants (Detke et al., 1997). Here, subchronic fluoxetine had no effect, while the 10 and 18 mg/kg/day doses of chronic fluoxetine increased swimming and reduced immobility in Balb/cJ mice. Furthermore, similar effects are shown using different classes of antidepressants (e.g. tricyclics). ANCOVA showed that the nonsignificant increases in locomotion induced by fluoxetine found in the open field were not responsible for the increases in swimming in either experiment. Previous reports have shown that 10 mg/kg fluoxetine also reduces immobility acutely in Balb/cJ mice (Lucki et al., 2001). The lack of effect of subchronic fluoxetine treatment (days 6-7) on swimming should not have resulted from lower plasma fluoxetine levels compared to chronically treated mice, since steady state blood levels have been achieved by at least day 5 with administration of fluoxetine in the drinking water (Santarelli et al., 2000). Rather, the high peak levels of fluoxetine resulting from acute injection are likely responsible for the sensitivity of Balb/cJ mice to acute treatment. Acute injection of antidepressant, which results in sharp peaks of drug in plasma, does not accurately model the initiation of antidepressant treatment in patients. Fluoxetine was administered in the drinking water to better mimic the patient's therapeutic regimen. Acute administration of 10 mg/kg/day fluoxetine in the drinking water should also produce no effect in the modified FST in Balb/cJ mice. The effects induced here in the modified FST by chronic (>21 days), but not subchronic (5 days), fluoxetine treatment may involve increases in neural plasticity and growth-related events (D'Sa and Duman, 2002).

Balb/cJ mice exhibited sensitivity to chronic, but not subchronic, treatment with fluoxetine treatment in the NIH test. In the novel cage, all three doses reduced the latency to consume milk relative to control and increased consumption in the first five minutes. Additionally, the 18 and 25 mg/kg/day doses decreased difference scores for latency (home vs. novel) relative to control. Only the 25 mg/kg/day dose increased consumption in the first five minutes in the home cage; however, this dose resulted in serum levels that are over twice the maximum levels found in patients taking 20-80 mg/day Prozac (Koran et al., 1996). This dose likely produced nonspecific effects. Overall, the 18 mg/kg/day dose of chronic fluoxetine showed maximal anxiolytic effects in the NIH paradigm. The NIH paradigm might also assess fluoxetine's effects on hedonic processes in addition to anxiety. The intake of palatable fluids, including sucrose and saccharin solutions, has been used as a measure of reward sensitivity (Willner, 1997; Le Pen et al., 2002). In such paradigms, reduced drinking is thought to reflect anhedonia, a core symptom of depression in the current diagnostic system, DSM-IV (American Psychiatric Association, 1994). Due to the high palatability of sweetened-condensed milk, reduced latency and increased consumption induced by chronic fluoxetine treatment might reflect a reduction in anhedonia in addition to anxiety in the NIH paradigm. The NIH paradigm offers advantages over previous hypophagia-based models. The use of a familiar and highly palatable snack instead of chow makes food deprivations unnecessary. Multiple dependent measures are applied identically in the control (home) and experimental (novel) conditions.

Several neuropsychiatric populations, including schizophrenia patients, exhibit deficits in PPI compared to healthy controls. PPI is theorized to provide an operational measure of sensorimotor gating, which is a form of central nervous system inhibition that filters out extraneous sensory, cognitive, and motor information to permit mental and behavioral integration. For example, deficient PPI correlates significantly with measures of distractability (Karper et al., 1996) and thought disorder (Perry et al., 1999) in schizophrenia patients. The PPI paradigm has been used most often to model in animals the deficient sensorimotor gating observed in schizophrenia (Geyer et al., 2001). Numerous studies have examined the mechanisms for typical and atypical antipsychotics to prevent PPI disruptions induced by direct or indirect dopamine agonists and NMDA receptor antagonists (Swerdlow and Geyer, 1993; Bakshi and Geyer, 1997, 1998; Geyer et al., 2001). However, PPI deficits are also observed in patients with obsessive compulsive disorder (Swerdlow et al., 1993; Braff et al., 2001), and panic disorder (Ludewig et al., 2002); SSRIs are first-line drug therapies for these disorders. Thus, the effects of chronic antidepressant treatment on drug-induced PPI deficits were investigated for the first time. All doses of chronic, but not subchronic, fluoxetine treatment prevent the PPI-disruptive effects of RU24969, a serotonin 1A/1B agonist. Two to three weeks of treatment with SSRIs are required to desensitize terminal 5-HT1B receptors (Blier, 2001). Additionally, RU24969 disrupts PPI via activation of the 5-HT1B receptor in mice (Dulawa et al., 1998; Dulawa et al., 2000). Thus, the desensitization of specific populations of 5-HT1B receptors, which reduce PPI when activated, might underlie the PPI-protective effect of chronic fluoxetine observed here. Further, the PPI paradigm may be exploited to identify the neural substrates underlying the sensorimotor gating deficits in specific anxiety-disordered populations.

The findings introduce four novel effects of chronic, but not subchronic, antidepressant treatment in the anxious Balb/cJ strain. These models are easy to use, appear reliable, and assess several different domains affected by antidepressant treatment: anxiety, depression, and sensorimotor gating.

### REFERENCES

Bakshi V P, Geyer M A (1997) Phencyclidine-induced deficits in prepulse inhibition of startle are blocked by prazosin, an alpha-1 noradrenergic antagonist. J Pharmacol Exp Ther 283:666-674.
Bakshi V P, Geyer M A (1998) Multiple limbic regions mediate the disruption of prepulse inhibition produced in rats by the noncompetitive NMDA antagonist dizocilpine. J Neurosci 18:8394-8401.
Barr A M, Markou A, Phillips A G (2002) A ‘crash’ course on psychostimulant withdrawal as a model of depression. Trends Pharmacol Sci 23:475-482.
Barr L C, Heninger G R, Goodman W, Charney D S, Price L H (1997) Effects of fluoxetine administration on mood response to tryptophan depletion in healthy subjects. Biol Psychiatry 41:949-954.
Belzung C, Le Pape G (1994) Comparison of different behavioral test situations used in psychopharmacology for measurement of anxiety. Physiol Behav 56:623-628.
Blier P (2001) Pharmacology of rapid-onset antidepressant treatment strategies. J Clin Psychiatry 62 Suppl 15:12-17.
Blier P (2003) The pharmacology of putative early-onset antidepressant strategies. Eur Neuropsychopharmacol 13:57-66.
Bodnoff S R, Suranyi-Cadotte B, Aitken D H, Quirion R, Meaney M J (1988) The effects of chronic antidepressant treatment in an animal model of anxiety. Psychopharmacology (Berl) 95:298-302.
Borsini F, Lecci A, Sessarego A, Frassine R, Meli A (1989) Discovery of antidepressant activity by forced swimming test may depend on pre-exposure of rats to a stressful situation. Psychopharmacology (Berl) 97:183-188.
Bouwknecht J A, Paylor R (2002) Behavioral and physiological mouse assays for anxiety: a survey in nine mouse strains. Behav Brain Res 136:489-501.
Braff D L, Geyer M A, Swerdlow N R (2001) Human studies of prepulse inhibition of startle: normal subjects, patient groups, and pharmacological studies. Psychopharmacology (Bern) 156:234-258.
Caccia S, Cappi M, Fracasso C, Garattini S (1990) Influence of dose and route of administration on the kinetics of fluoxetine and its metabolite norfluoxetine in the rat. Psychopharmacology (Berl) 100:509-514.
Cryan J F, Lucki I (2000) Antidepressant-like behavioral effects mediated by 5-Hydroxytryptamine(2C) receptors. J Pharmacol Exp Ther 295:1120-1126.
Cryan J F, Markou A, Lucki I (2002a) Assessing antidepressant activity in rodents: recent developments and future needs. Trends Pharmacol Sci 23:238-245.
Cryan J F, Page M E, Lucki I (2002b) Noradrenergic lesions differentially alter the antidepressant-like effects of reboxetine in a modified forced swim test. Eur J Pharmacol 436:197-205.
D'Sa C, Duman R S (2002) Antidepressants and neuroplasticity. Bipolar Disord 4:183-194.
Detke M J, Johnson J, Lucki I (1997) Acute and chronic antidepressant drug treatment in the rat forced swimming test model of depression. Exp Clin Psychopharmacol 5:107-112.
Dulawa S C, Hen R, Scearce-Levie K, Geyer M A (1998) 5-HT1B receptor modulation of prepulse inhibition: recent findings in wild-type and 5-HT1B knockout mice. Ann N Y Acad Sci 861:79-84.
Dulawa S C, Grandy D K, Low M J, Paulus M P, Geyer M A (1999) Dopamine D4 receptor-knock-out mice exhibit reduced exploration of novel stimuli. J Neurosci 19:9550-9556.
Dulawa S C, Gross C, Stark K L, Hen R, Geyer M A (2000) Knockout mice reveal opposite roles for serotonin 1A and 1B receptors in prepulse inhibition. Neuropsychopharmacology 22:650-659.
Gelfin Y, Gorfine M, Lerer B (1998) Effect of clinical doses of fluoxetine on psychological variables in healthy volunteers. Am J Psychiatry 155:290-292.
Geyer M, Markou A (1995) Animal Models of psychiatric disorders. In: Psychopharmacology: The fourth generation of progress. (Bloom FE KD, ed). New York: Raven Press.
Geyer M, Markou A (2002) The role of preclinical models in the development of psychotropic drugs. In: Neuropsychopharmacology: The fifth generation of progress. (K L Davis DC, J T Coyle, C Nemeroff, ed), pp 446-455. Philadelphia: Lippincott Williams & Wilkins.
Geyer M A, Braff D L (1987) Startle habituation and sensorimotor gating in schizophrenia and related animal models. Schizophr Bull 13:643-668.
Geyer M A, Krebs-Thomson K, Braff D L, Swerdlow N R (2001) Pharmacological studies of prepulse inhibition models of sensorimotor gating deficits in schizophrenia: a decade in review. Psychopharmacology (Berl) 156:117-154.
Geyer M A (2002) The role of preclinical models in the development of psychotropic drugs. In: Neuropsychopharmacology: The fifth generation of progress. (K L Davis DC, J T Coyle, C Nemeroff, ed), pp 446-455. Philadelphia: Lippincott Williams & Wilkins.
Graham F K (1975) Presidential Address, 1974. The more or less startling effects of weak prestimulation. Psychophysiology 12:238-248.
Hiemke C, Hartter S (2000) Pharmacokinetics of selective serotonin reuptake inhibitors. Pharmacol Ther 85:11-28.
Hirschfeld R,. Weisssman M (2002) Risk factors for major depression and bipolar disorder. In: Neuropsychopharmacology: The fifth generation of progress (K L Davis DC, J T Coyle, C Nemeroff, ed), pp 1018-1025. Philadelphia: Lippincott Williams & Wilkins.
Karper L P, Freeman G K, Grillon C, Morgan C A, 3rd, Charney D S, Krystal J H (1996) Preliminary evidence of an association between sensorimotor gating and distractibility in psychosis. J Neuropsychiatry Clin Neurosci 8:60-66.
Kokkinidis L, Zacharko R M, Predy P A (1980) Post-amphetamine depression of self-stimulation responding from the substantia nigra: reversal by tricyclic antidepressants. Pharmacol Biochem Behav 13:379-383.
Koran L M, Cain J W, Dominguez R A, Rush A J, Thiemann S (1996) Are fluoxetine plasma levels related to outcome in obsessive-compulsive disorder? Am J Psychiatry 153:1450-1454.
Le Pen G, Gaudet L, Mortas P, Mory R, Moreau J L (2002) Deficits in reward sensitivity in a neurodevelopmental rat model of schizophrenia. Psychopharmacology (Berl) 161:434-441.
Lucki I, Dalvi A, Mayorga A J (2001) Sensitivity to the effects of pharmacologically selective antidepressants in different strains of mice. Psychopharmacology (Berl) 155:315-322.
Ludewig S, Ludewig K, Geyer M A, Hell D, Vollenweider F X (2002) Prepulse inhibition deficits in patients with panic disorder. Depress Anxiety 15:55-60.
Markou A, Hauger R L, Koob G F (1992) Desmethylimipramine attenuates cocaine withdrawal in rats. Psychopharmacology (Berl) 109:305-314.
Merikangas K, Pine D (2002) Genetic and other vulnerability factors for anxiety and stress disorders. In: Neuropsychopharmacology: The fifth generation of progress. (K L Davis DC, J T Coyle, C Nemeroff, ed), pp 868-882. Philadelphia: Lippincott Williams & Wilkins.
Moreau J L, Jenck F, Martin J R, Mortas P, Haefely W E (1992) Antidepressant treatment prevents chronic unpredictable mild stress-induced anhedonia as assessed by ventral tegmentum self-stimulation behavior in rats. Eur Neuropsychopharmacol 2:43-49.
Perry W, Geyer M A, Braff D L (1999) Sensorimotor gating and thought disturbance measured in close temporal proximity in schizophrenic patients. Arch Gen Psychiatry 56:277-281.
Prut L, Belzung C (2003) The open field as a paradigm to measure the effects of drugs on anxiety-like behaviors: a review. Eur J Pharmacol 463:3-33.
Robertson H A (1979) Benzodiazepine receptors in “emotional” and “non-emotional” mice; comparison of four strains. Eur J Pharmacol 56:163-166.
Santarelli L, Gross C, Monckton J, Dulawa S, Lee J, Saxe M, Hen R (2000) The 5-HT1A receptor is required for the effect of chronic fluoxetine on behavior and neurogenesis. Soc Neurosci Abstr.
Shephard R A, Broadhurst P L (1982) Hyponeophagia and arousal in rats: effects of diazepam, 5-methoxy-N,N-dimethyltryptamine, d-amphetamine and food deprivation. Psychopharmacology (Berl) 78:368-372.
Suckow R F, Zhang M F, Cooper T B (1992) Sensitive and selective liquid-chromatographic assay of fluoxetine and norfluoxetine in plasma with fluorescence detection after precolumn derivatization. Clin Chem 38:1756-1761.
Swerdlow N R, Geyer M A (1993) Clozapine and haloperidol in an animal model of sensorimotor gating deficits in schizophrenia. Pharmacol Biochem Behav 44:741-744.
Swerdlow N R, Benbow C H, Zisook S, Geyer M A, Braff D L (1993) A preliminary assessment of sensorimotor gating in patients with obsessive compulsive disorder. Biol Psychiatry 33:298-301.
Tang X, Orchard S M, Sanford L D (2002) Home cage activity and behavioral performance in inbred and hybrid mice. Behav Brain Res 136:555-569.
Welker W (1957) “Free” versus “forced” exploration of a novel situation by rats. Psychol Rep 3:95-108.
Willner P (1997) Validity, reliability and utility of the chronic mild stress model of depression: a 10-year review and evaluation. Psychopharmacology (Berl) 134:319-329.

1. A method for determining whether an agent reduces anxiety and/or depression in a depressed, non-human subject, comprising:
(a) determining the subject's rate of engagement in a pleasurable activity in a novel environment, wherein at the time the subject is introduced to the novel environment, the subject has had the agent administered to it over a suitable period of time;
(b) comparing the subject's rate of engagement determined in step (a) with the subject's rate of engagement in the pleasurable activity determined prior to the subject's introduction to the novel environment when the subject is in a familiar environment and has had the agent administered to it for a suitable period of time, so as to determine the difference in rates of engagement; and
(c) comparing the difference in rates of engagement determined in step (b) with the difference in rates of engagement determined in a depressed, non-human subject to which the agent has not been administered, whereby the difference determined in step (b) being less than the difference determined in the absence of the agent indicates that the agent reduces anxiety and/or depression in a depressed, non-human subject.

2. The method of claim 1, wherein the subject is a mammal.

3. The method of claim 2, wherein the mammal is a mouse.

4. The method of claim 3, wherein the mouse is a Balb/cJ mouse.

5. The method of claim 1, wherein the agent is selected from the group consisting of a MAO inhibitor, a NK1 antagonist, a CRF1 antagonist, a vasopression1B antagonist, a glucocorticoid receptor antagonist, a 5-HT1B receptor antagonist and a lithium mood stabilizer.

6. The method of claim 1, wherein the agent is selected from the group consisting of desipramine, venlafaxine, reboxetine and fluoxetine.

7. The method of claim 1, wherein the agent is an electroconvulsive shock or transcranial magnetic stimulation.

8. The method of claim 1, wherein the subject's rate of engagement in the pleasurable activity is the speed with which the subject commences the activity.

9. The method of claim 1, wherein the subject's rate of engagement in the pleasurable activity is the speed with which the subject carries out the activity once the activity has commenced.

10. The method of claim 1, wherein the pleasurable activity is selected from the group consisting of eating, drinking, sexual activity, object exploration, and the receipt of a pleasurable electrical stimulus.

11. The method of claim 1, wherein the environment is a cage.

12. The method of claim 11, wherein the novel and familiar environments differ from one another via a difference in bedding, lighting and/or noise level.

13. The method of claim 1, wherein the difference in rates of engagement determined in step (c) is at least 50%.

14. The method of claim 13, wherein the difference in rates of engagement determined in step (c) is at least 100%.

15. The method of claim 14, wherein the difference in rates of engagement determined in step (c) is at least 150%.

16. The method of claim 1, wherein the suitable period of time is at least one week.

17. The method of claim 1, wherein the suitable period of time is at least two weeks.

18. The method of claim 1, wherein the suitable period of time is at least four weeks.

19. The method of claim 1, further comprising the step of performing the method a plurality of times using different dosages of the agent and comparing the differences in rates of engagement, so as to determine which of the dosages used best reduces anxiety and/or depression in the subject.

20. The method of claim 1, further comprising the step of comparing the differences in rates determined in step (c) for a subject to which the agent has been administered with the differences in rates determined in step (c) for a subject to which fluoxetine has been administered in the same dose as the agent, so as to determine whether the agent reduces anxiety and/or depression as well as, or better than, fluoxetine.

21. The method of claim 1, further comprising the step of comparing (i) the minimum period of time over which the agent must be administered to reduce anxiety and/or depression in the subject with (ii) the minimum period of time over which fluoxetine must be administered to reduce anxiety and/or depression in the subject, so as to determine whether the agent reduces anxiety and/or depression in a subject via administration over a time period as short as, or shorter than, that required for fluoxetine to achieve the same result.

22. A method of treating anxiety in a subject afflicted with that disorder comprising administering to the subject a therapeutically effective amount of an anxiety-reducing agent identified by the method of claim 1.

23. A method of treating depression in a subject afflicted with that disorder comprising administering to the subject a therapeutically effective amount of a depression-reducing agent identified by the method of claim 1.
